(19) United States
(12) Patent Application Publication
(10) Pub. No.: US 2020/0248172 A1
(43) Pub. Date: Aug. 6, 2020

(54) SOIL-BASED DNA EXTRACTION

(71) Applicant: Winfield Solutions, LLC, Arden Hills, MN (US)

(72) Inventor: Elizabeth Buescher, Gilbert, IA (US)

(21) Appl. No.: 16/855,589

(22) Filed: Apr. 22, 2020

Related U.S. Application Data
(63) Continuation-in-part of application No. 16/165,847, filed on Oct. 19, 2018.

(51) Int. Cl. C12N 15/10; C12N 9/42

(52) U.S. Cl. CPC C12N 15/1006; C12N 9/2437

(57) ABSTRACT

Methods of extracting microbial DNA from soil involve lysing microbial cells contained within the soil by mixing the soil with one or more enzymes, sonicating the soil, or both. Methods further involve precipitating DNA lysed from the microbial cells using cold isopropanol to form a DNA pellet, washing non-DNA debris from the DNA pellet, and re-suspending the DNA in a resuspension buffer. Example methods may involve extracting microbial DNA from a plurality of soil samples containing soybean cyst nematodes, fungal spores, and other microbial species armed with fibrous and/or proteinaceous materials. Such methods can involve adding soil slurry samples into separate wells within a multi-well plate, lysing microbial cells within each sample using enzymatically- and/or mechanically-enhanced lysis techniques, precipitating the microbial DNA released from the cells, washing non-DNA debris from the resulting DNA pellets, and re-suspending the DNA for further analysis.

### CROSS-REFERENCE TO RELATED APPLICATION

This application is a continuation-in-part of U.S. patent application Ser. No. 16/165,847 filed on Oct. 19, 2018, and entitled “Soil-Based DNA Extraction,” the contents of which are herein incorporated by reference in their entirety.

### TECHNICAL FIELD

Implementations relate to methods of extracting deoxyribonucleic acid from soil. Particular implementations involve scalable methods of extracting microbial DNA from small to moderately sized soil samples.

### BACKGROUND

Genomic DNA from microbial populations present in soil can be extracted and analyzed to acquire species-specific data reflecting a variety of environmental processes. For example, soil DNA data can arm plant producers with the information needed to identify and quantify microbes present within their soil samples impacting specific disease pressures and vital nutrient cycles. These data can then be used to inform prediction models regarding overall crop yield within a particular geographic location. Understanding the microbial makeup of soil leads to smarter, more effective crop production.

Various approaches to extracting and quantifying DNA from soil samples have been developed, most of which utilize commercial kits containing proprietary extraction devices and reagents. While such kits can be used to generate large amounts of DNA, large yields are not necessarily desirable for certain applications, the DNA concentrations may be low, and the kit components and corresponding protocols are usually expensive and not amenable to user-specific adjustments. The kits are also often incapable of reliably extracting DNA from difficult-to-access microbes, such as soybean cyst nematodes. As such, the kits may produce inaccurate data despite their expense. The kits may also lack scalability, and may frequently generate large amounts of wasted products. In addition, the kits can be labor intensive, thus requiring significant time and effort to extract DNA, and protocols developed for other sample types, e.g., plant or human tissue, are not compatible with soil-based DNA extraction techniques. New, standardized approaches to extracting and quantifying microbial DNA from a wide variety of soil samples are thus desired.

### BRIEF DESCRIPTION OF THE DRAWINGS

FIG. 1 is a flow diagram of a method performed in accordance with principles of the present disclosure.

FIG. 2 is a flow diagram of another method performed in accordance with principles of the present disclosure.

FIG. 3 is a flow diagram of another method performed in accordance with principles of the present disclosure.

### SUMMARY

Implementations provide methods of extracting genomic DNA from soil. Specific embodiments involve methods and reagents for extracting genomic DNA from microbial populations included in small to moderately sized soil samples. The methods may be readily scaled according to user preferences, and may be implemented using standard laboratory equipment and common laboratory reagents without the need for commercial kits. The methods may also be designed to consistently quantify DNA from various microbial species, such as soybean cyst nematodes, known to impact common disease pressures and/or nutrient cycles. Such methods, which may be high-throughput and feature enhanced lysis techniques, can improve the likelihood of detecting and accurately quantifying a greater number of microbial species evasive to detection, thereby revealing a greater number of promising seed treatment options for producers. Example methods may involve lysing microbial cells contained in a soil-water slurry to release DNA therefrom via enzymatic and/or mechanical techniques. Methods may also involve binding the released DNA using a silica binding solution or precipitating the released DNA with isopropanol. After DNA binding and/or precipitation, example methods may involve washing non-DNA debris from the DNA to isolate the targeted DNA in pure form, and resuspending the isolated DNA for continued analysis. The final DNA samples may be free or substantially free of humic acid and thus highly pure, thereby improving the effectiveness of PCR-based amplification and analysis.

In accordance with principles of the present disclosure, a method of extracting DNA from a soil sample may involve lysing microbial cells within the soil sample by mixing the soil sample with at least one enzyme and/or by sonicating the soil sample. The method may further involve precipitating DNA released from the lysed microbial cells to form a precipitated DNA pellet, washing non-DNA debris from the precipitated DNA pellet to isolate microbial DNA, and re-suspending the isolated microbial DNA in a re-suspension buffer.

In some examples, mixing the soil sample with at least one enzyme may involve mixing the soil sample with cellulase. In some embodiments, mixing the soil sample with at least one enzyme may involve mixing the soil sample with a mixture of chitinase and cellulase. In some examples, sonicating the soil sample may involve ultrasonicating the soil sample with a sonication buffer in an ice bath. In some embodiments, ultrasonicating the soil sample may involve ultrasonicating the soil sample with an ultrasonicator probe ranging in diameter from about ⅛ in. to about ¼ in. In some examples, ultrasonicating the soil sample may involve ultrasonicating the soil sample according to a pulsating sonication program. In some embodiments, the pulsating sonicating program may comprise interleaving segments of pulsing and rest, the pulsing segments implemented at about 50 to about 75% amplitude.

Example methods may further involve adding a cTAB solution to the sonicated soil sample. In some embodiments, the cTAB solution can be pre-warmed to a temperature ranging from about 50° C. to about 65° C. In some examples, the cTAB solution may comprise about 20% pure cTAB.

Example methods may further involve wet-sieving the soil sample prior to lysing the microbial cells. In some embodiments, wet-sieving the soil sample may involve mixing the soil sample with water to form a soil slurry and passing the soil slurry through one or more sieves. In some examples, the one or more sieves may comprise two stacked sieves having different pore sizes.

In some embodiments, the soil sample can comprise between about 250 and about 750 mg of soil, inclusive. In some examples, the soil sample can comprise a 2 mL mixture of soil and water. In some embodiments, washing non-DNA debris from the precipitated DNA pellet may involve re-suspending the pellet in a wash buffer and centrifuging the re-suspension. In some examples, precipitating DNA released from the lysed microbial cells can involve admixing isopropanol with the lysed microbial cells. In some embodiments, the isopropanol can be maintained at a temperature of about −20° C. Example methods can further involve incubating the lysed microbial cells and the isopropanol for at least about 2 hours. Embodiments can also involve centrifuging the lysed microbial cells and the isopropanol at about 2500×g for about 7 to about 10 minutes after incubating.

### DETAILED DESCRIPTION

The soil-based DNA extraction methods provided herein can advantageously utilize a smaller amount of soil compared to preexisting methods and may reduce, minimize or eliminate humic acid content within the soil samples during extraction, thereby improving final DNA purity. The methods may also be high-throughput and scaled as desired to meet user preferences, making the methods adaptable to a variety of different applications, e.g., research and industrial. The methods may also be capable of effectively lysing microbes protected by a variety of fibrous and proteinaceous materials. For example, cyst-producing nematodes have cellulose-containing polymers that protect the cysts' eggs. Fungal spores are similarly protected by chitin. Disclosed embodiments may utilize specific enzymes and/or mechanical techniques, e.g., ultrasonication, to successfully lyse cysts, eggs contained within cysts, fungal spores, and other microbes armed with various forms of physical protection. As a result, the final genomic DNA products generated via the methods described herein may include a comprehensive representation of the microbial populations present within a given soil sample. Such DNA products may also be highly concentrated and pure, making downstream DNA amplification and characterization easier and more accurate. The equipment required to implement the methods may be standard equipment typically found in most research or production laboratories. Specialized devices, such as the disposable bead tubes, membranes and spin columns frequently included in commercial kits, are not required to perform the methods herein, thereby providing additional benefits related to cost expenditures, accessibility and waste.

Extraction methods provided herein may involve multiple stages, which may generally include: lysis, DNA precipitation, DNA binding, washing, resuspension, and/or elution stages, with each stage comprising a plurality of steps. Methods may also involve a pre-processing stage. The particular combination of processing stages implemented may vary, depending in part on the suspected composition of the soil sample(s) being evaluated and/or the particular microbial species of interest to the testing party. For example, soil samples containing populations of microbes protected by chitin and/or cellulose may be subjected to enzymatic and/or mechanically-enhanced lysis techniques followed by isopropanol-driven DNA precipitation, without DNA-binding. Alternatively, certain embodiments may involve sonication-powered lysis followed by silica-based DNA binding. Embodiments may also involve a combination of the aforementioned examples.

The lysis stage breaks apart the microbial cells included within the soil samples, thereby exposing the internal cellular components, including total genomic DNA. The DNA precipitation stage concentrates the microbial DNA released from the lysed cells. The DNA binding stage causes DNA molecules released from the lysed cells to bind with silica particles included in a DNA binding solution. The washing stage removes extraneous, i.e., non-DNA, soil debris, residual chemicals required in DNA extraction, and cellular components, including lipids, proteins and organelles, etc. The re-suspension and/or elution stage resuspends the isolated DNA in an elution buffer for quantification and follow-up analysis.

Reagents:

Prior to performing one or more of the aforementioned stages, one or more of the reagents utilized in the extraction process may be prepared and optionally stored. Reagents utilized according to the methods herein may include, but are not limited to: NaCl, Tris-EDTA, sodium acetate, a wash buffer, chitinase, cellulase, a chitinase/cellulase mixture, hydrogen peroxide, a DNA binding buffer, a sonication buffer, an ultrasonication buffer, a cTAB solution, a DNA elution buffer, and/or a DNA binding solution. The specific volumes, concentrations and/or pH levels provided herein for each reagent may be for example purposes only, and should not be viewed as limiting. For example, the amount and/or properties of one or more reagents may be modified according to user preferences such that the disclosed methods are fully scalable. In some embodiments, for example, the volumes of one or more stock reagents may be multiplied to accommodate more plates utilized for an extraction. Stock solutions having higher concentrations than those disclosed below, for example, may be prepared and diluted upon use. In addition, the total volume of each reagent can be readily adjusted depending on the number of soil samples simultaneously used for DNA extraction.

In some examples, the NaCl (sodium chloride) may be 5M (or about 5M), having a molecular weight of 58.44 g/mol. To prepare 5M NaCl, 146.1 grams of NaCl (or about 146.1 grams) may be mixed with distilled water to reach a total volume of 500 mL (or about 500 mL).

In various examples, 10× Tris-EDTA (ethylenediamine tetraacetic acid or “TE”) (or about 10×) may be prepared by mixing 100 mL of 1M Tris-HCl, pH 8.0 (or about 100 mL) with 20 mL of 0.5M EDTA, pH 8.0 (or about 20 mL of about 0.5M EDTA) and adding distilled water to reach a total volume of 1 L (or about 1 L).

The sodium acetate may be 3M (or about 3M), having a molecular weight of 82.03 g/mol. To prepare the sodium acetate, 123.05 grams of sodium acetate (or about 123.05 grams) can be mixed with water to reach a total volume of 500 mL (or about 500 mL). The pH may then be adjusted to 5.2 (or about 5.2), for example using glacial acetic acid.

The chitinase/cellulase enzymatic mixture can formed by obtaining 250 mg of chitinase/cellulase enzyme mix (or about 250 mg) and adding 600 mM potassium chloride buffer (or about 600 mM) up to a total volume of 50 mL (or about 50 mL).

The cellulase can be formed by obtaining 250 mg of cellulase powder (or about 250 mg) and adding 50 mM acetate buffer (or about 50 mM) up to a total volume of 50 mL (or about 50 mL).

The chitinase can be formed by obtaining 250 mg of chitinase powder (or about 250 mg) and adding 50 mM acetate buffer (or about 50 mM) up to a total volume of 50 mL (or about 50 mL).

The hydrogen peroxide may be 200 mM (or about 200 mM), and may be formed by obtaining 818 μL of 30% hydrogen peroxide (or about 818 μL of about 30% hydrogen peroxide) and adding deionized water up to a total volume of 1 L.

One embodiment of the wash buffer can include 0.05M NaCl (or about 0.05M) and may comprise 90% ethanol (or about 90%). The wash buffer may be prepared by mixing 3 mL of 5M NaCl (or about 3 mL of about 5M NaCl) with 270 mL of 100% ethanol (or about 270 mL of about 100% ethanol) and adding distilled water to reach a total volume of 300 mL (or about 300 mL). Another embodiment of the wash buffer may comprise 70% ethanol (or about 70%). A 70% ethanol wash buffer can be prepared by obtaining 700 mL of 100% ethanol and adding distilled water to reach a total volume of 1 L.

The DNA binding buffer may comprise 6M guanidine thiocyanate (or about 6M), having a molecular weight of 118.2 g/mol. To prepare the DNA binding buffer, 70.92 grams of the 6M guanidine thiocyanate (or about 70.92 grams of about 6M guanidine thiocyanate) can be mixed with distilled water, reaching a total volume of 100 mL (or about 100 mL).

The sonication buffer can comprise 0.5M NaCl (or about 0.5M), 3% PVP (or about 3%) (polyvinylpyrrolidone, weight 40,000), 10× TE (or about 10×), RNase A, and cTAB (cetrimonium bromide), and it may be prepared by mixing 25 mL of 5M NaCl (or about 25 mL of about 5M NaCl) with 7.5 grams of PVP (or about 7.5 grams), and 2% w/v cTAB (or about 2% w/v, which may equal about 5 grams cTAB), and adding 10× TE (or about 10×) up to a total volume of 250 mL (or about 250 mL). The RNase A may be added just prior to use of the sonication buffer. In embodiments, about 50 μL of RNase A at a concentration of 5 μg/μL may be added per 50 mL of sonication buffer. The sonication buffer may specifically exclude additional reagents, including proteinase K, SDS (sodium dodecyl sulfate), and various enzymes, e.g., lysozymes and glusulase, thereby further simplifying the processes described herein.

The ultrasonication buffer can include 0.5M NaCl (or about 0.5M), 3% PVP (or about 3%) (polyvinylpyrrolidone, weight 40,000), and 10× TE (or about 10×). The ultrasonication buffer can be prepared by mixing 25 mL of 5M NaCl (or about 25 mL of about 5M NaCl) with 7.5 grams of PVP (or about 7.5 grams), and adding 10× TE (or about 10×) up to a total volume of 250 mL (or about 250 mL). Embodiments of the ultrasonication buffer may specifically exclude certain additional reagents, including cellulase and/or chitinase, for example.

The DNA elution buffer can comprise 0.1× Tris-EDTA (or about 0.1×), which may be prepared by mixing 2 mL of 10× Tris-EDTA (or about 2 mL of about 10× Tris-EDTA) with 48 mL of distilled water (or about 48 mL).

The DNA binding solution utilized according to some embodiments disclosed herein may include a combination of silica powder and water, and may replace the commercial binding solutions and associated devices commonly included in commercial kits. Preparation of the DNA binding solution can involve washing and suspending silica powder with water one or more times, e.g., 2, 3, 4 or more times. In one embodiment, about 800 mg of silica powder, e.g., CELITE® 545, can be poured into a 50 mL centrifugation tube, e.g., a BD FALCON™ tube, such that the powder reaches to about the 2.5 mL mark on the tube. About 30 mL of water, which may be distilled, can be added to the tube and mixed with the silica powder by vigorously shaking and/or vortexing. The resulting slurry may then be allowed to settle for approximately 15 minutes, or at least until the powder and water separate. The water can then be decanted and another aliquot of about 30 mL of water added to the silica powder. The aforementioned washing steps, i.e., mixing, separating and decanting, can then be repeated two or more times. After the final washing step, the silica powder can be resuspended in a fresh aliquot of water. The volume of water used to resuspend the silica may vary. In some examples, the volume of water may be approximately equal to the volume of silica, such that the ratio of silica to water is about 1:1 and the resuspension fills about 5 mL of the centrifuge tube. The finished DNA binding solution can be stored at about room temperature, e.g., 20-25° C., until further use. The DNA binding solution may comprise silica and water, only, without any additional components, such as potassium iodide. The simplified solution may provide yet another advantage over preexisting systems.

Immediately prior to use, the DNA binding solution can be resuspended by manually shaking, pipetting and/or vortexing. About 200 μL of the DNA binding solution can then be transferred, e.g., pipetted, into each of one or more wells defined by a multi-well plate. The capacity of each well can be 8 mL in some embodiments, and the number of wells included in the plate may be 24, although the number of wells is not critical. For example, the capacity of each well in a 96-well plate can be 2 mL. Other wells within a multi-well plate may have a 10 mL capacity. The number of wells needed, whether in one plate or multiple, depends on the number of soil samples evaluated, as each soil sample is assigned to one well. Because the methods disclosed herein do not rely on predefined kits, which can only be used to process a limited number of samples simultaneously, the number of soil samples can be scaled up or down as desired by a user, limited only by the number of multi-well plates available and/or the number of plates that can fit in a centrifuge. For example, DNA from about 84 separate soil samples can be simultaneously extracted according the methods herein, such that about 500 samples can be reasonably extracted by one user in one day regardless of the particular lysis, DNA-binding and/or DNA-precipitation techniques employed. This marks a significant improvement over preexisting methods, which may be limited to about 25 samples per user per day. In addition, the multi-well plates can be washed and reused. For instance, plates may be re-washed after each extraction.

During the transfer of DNA binding solution to each well, the remaining stock solution may be periodically agitated to maintain uniform distribution of the silica in the suspension. To each well containing DNA binding solution, 1 mL of the DNA binding buffer can then be added and used to resuspend the silica. Resuspension can be achieved by pipetting (repeatedly up and down) the mixture of DNA binding solution and buffer.

Pre-Processing Stage:

Methods herein are not limited to a particular soil composition type, and thus may be amenable to soils of varying moisture levels, including soil with various levels of sand, silt, clay, peat, organic matter, etc. One or more soil pre-processing steps may be implemented before the lysis stage. For example, unlike preexisting approaches that may utilize dry soil, methods herein may utilize slurries of soil and water. As such, a pre-processing step can involve wet-milling the soil sample(s) used for DNA extraction. Wet-milling the soil may involve mixing, e.g., blending, the soil with water to form a slurry. Replicates of each soil sample can be prepared, along with one or more control samples. The amount of soil used to prepare each slurry sample may vary, and may be less than amounts required for preexisting DNA extraction protocols, which may recommend about 10 grams of soil per sample. In various embodiments, the amount of soil used to create a slurry for each sample can range from about 250 mg to about 1 gram, about 350 mg to about 750 mg, about 450 mg to about 550 mg, about 475 mg to about 525 mg, or about 500 mg. In some examples, dried, ground soil may still be used. Such examples may be performed with some variations, e.g., without the use of silica as a binding agent.

Lysis Stage:

The lysis stage bursts, punctures and otherwise breaks apart microbial cells present within the soil samples, thereby exposing the internal cellular components, including total genomic DNA, organelles, proteins, etc. Different lysis techniques may be employed in accordance with the disclosed embodiments, which may depend at least in part on the starting soil composition. For example, the lysis stage may comprise enzymatic lysis, mechanical lysis, or both. Enzymatically and/or mechanically-enhanced lysis techniques can have a lysis efficiency exceeding 90%, e.g., about 97%. This marks a significant improvement over preexisting lysis techniques, especially when employed to lyse tough microbial organisms, such as cysts, cyst eggs and/or fungal spores. Such preexisting, less-harsh approaches often produced lysis efficiencies of only about 50%.

Within the context of the present disclosure, “enhanced” lysis may be defined as lysis performed using one or more enzymes, such as chitinase and/or cellulase, lysis performed using one or more mechanical techniques, such as ultrasonication, or lysis performed using a combination of two or more of the aforementioned techniques. As defined herein, “ultrasonication” may be implemented using single- or multi-probe ultrasonicator(s), e.g., 24-probe ultrasonicator(s), in conjunction with the ultrasonication buffer detailed above. “Sonication” may be implemented using a probe-less sonication apparatus, such as the 117V VWR® Ultrasonic Cleaner, in conjunction with the sonication buffer detailed above. Sonication may also be implemented in a heated water bath, which can activate any enzymes used during the lysis stage and eliminate the need to utilize comparatively harsh, probe-mediated ultrasonication. In some embodiments, enhanced lysis may be achieved via enzyme-aided sonication, such as sonication implemented before, after or concurrently with cellulase and/or chitinase digestion.

In one embodiment of the lysis stage, samples of each soil slurry, for example about 2 mL, can be pipetted into each well of the multi-well plate(s). To each well, 2.5 mL of sonication buffer can then be added and the plate(s) sonicated for about 30 seconds to about 2 minutes to lyse the cells within the slurry. In some embodiments, RNase A may be added to the sonication buffer just prior to sonication. For example, about 50 μL of RNase A at a concentration of 5 μg/μL may be added per 50 mL of sonication buffer, such that 12.5 μg of RNase A are added to each well upon addition of 2.5 mL sonication buffer. The sonication settings applied to the samples may vary. In one example, the plate(s) can be sonicated using a 117V VWR® Ultrasonic Cleaner. The inclusion of cTAB within the sonication buffer, for example at about 2% w/v, can precipitate humic acid and humic acid aggregate components during sonication, facilitating the removal of such substances, which may otherwise contaminate the isolated DNA and inhibit downstream analysis, e.g., PCR. After sonication, 1 mL of 3M sodium acetate can be added to each well, followed by optional mixing, and each plate placed in freezer set at −20° C. for 10 minutes. The plates may then be removed from the freezer and centrifuged at 4816×g (4700 rpm) for 5 minutes at room temperature, thereby separating the released cellular components and DNA (supernatant) from the solid soil debris. The lysis stage may be performed without the use of any metal beads and/or grinding. The aforementioned lysis stage may be considered “non-enhanced” within the context of the present disclosure.

In another embodiment of the lysis stage, which may be considered “enhanced” within certain embodiments of this disclosure, cysts present within the soil can be removed and lysed by first wet-sieving each soil slurry sample. Wet-sieving may generally involve mixing each soil sample with water to form a soil slurry and passing the slurry through one or more sieves. Particular embodiments may involve passing each slurry through one or more sieves stacked on top of each other. According to such embodiments, the stacked sieves may have different pore sizes, ranging from about 10 μm to about 80 μm.

For instance, successive samples of about 80 g and about 240 g of water may be used to sieve a given soil sample through successively stacked sieves having mesh pore ratings of 20 and 60 μm, respectively. All debris can then be removed from the 60-μm sieve and placed in a single well within a 24-well plate. The final volume within the well can be adjusted to about 2 mL. To quantify fungal spores, 2 mL of soil slurry can be pipetted into each well of the 24-well plate. To each well, 2.5 mL of sonication buffer and 500 μL of cellulase or chitinase/cellulase mixture can be added, along with 50 μL of 200 mM hydrogen peroxide. The plate can then be incubated in a 50° C. (121° F.) water bath for about 30 minutes to about 2 hours to activate the chitinase/cellulase mixture. After the incubation period, the plate can be subjected to a 3-minute sonication using a 117V VWR® Ultrasonic Cleaner. Optionally, 1 mL of 3M sodium acetate (pH 5.0) can be added to each well, and the plate placed in freezer set at −20° C. for 10 minutes. The plates may then be removed from the freezer and centrifuged at 2500×g for 7 minutes at room temperature (˜20° C.), to separate the released cellular components and DNA (supernatant) from the solid soil debris.

In another embodiment of the lysis stage, which may also be considered “enhanced” for the purposes of this disclosure, cysts present within the soil can likewise be removed by wet-sieving each soil slurry sample. Embodiments can involve successively sieving the slurry through sieves characterized by different pore sizes. For example, successive samples of about 80 g and about 240 g of water may be used to sieve a given soil sample through successively stacked sieves having mesh pore ratings of about 20 and about 60 μm, respectively. All debris can then be removed from the 60-μm sieve and placed in a single well within a 24-well plate. Optionally, debris collected on the 60-μm sieve can be crushed, for example using a drill press fitted with a rubber stopper. Debris can then be pressed through another 60-μm sieve and eggs contained in the cysts filtered through successive 200-μm and 500-μm sieves. Debris on the 500-μm sieve can then be removed from the filter and pipetted into a single well on a 24-well plate. Subsequent ultrasonication may then be implemented for the cyst eggs only.

Further pursuant to the enhanced lysis stage, fungal spores or other bacteria may be lysed by pipetting 2 mL of soil slurry into one or more wells of a 24-well plate. To each well containing pipetted slurry, 2.5 mL of ultrasonication buffer can be added. Each sample can then be ultrasonicated in an ice bath or at room temperature according to various sonication programs, which may comprise a consistent ultrasonication period or a programmed pulsation featuring interleaved periods of ultrasonication and rest. The parameters of each program, e.g., the length and intensity of each interleaving pulsation, along with the temperature at which ultrasonication is implemented, may depend on the particular microbial species targeted for lysis.

For example, for whole-cyst lysis and/or lysis implemented via 24 ultrasonicator probes, the samples can be ultrasonicated while submerged in an ice bath. For cyst egg lysis and/or lysis implemented via a single ultrasonicator probe, the samples can be ultrasonicated at room temperature or in an ice bath. For whole-cyst lysis and/or lysis implemented via a single ultrasonicator probe, a ¼ inch diameter ultrasonicator probe may be employed for a 14-minute pulsing ultrasonication program, which may involve repeated cycles of 2 minutes of pulsing and 30 seconds of rest, at 50-75% amplitude. In additional embodiments utilizing single-probe, whole-cyst lysis, a ⅛ inch diameter ultrasonicator probe may be employed for a 20-minute pulsing ultrasonication program, which may involve repeated cycles of 15 seconds of pulsing and 15 seconds of rest, at 75% amplitude. In specific embodiments utilizing single-probe, egg/spore lysis, a ¼ inch diameter ultrasonicator probe may be employed for a 3-minute ultrasonication, at 50-75% amplitude. In additional embodiments involving single-probe, egg/spore lysis, a ⅛ inch diameter ultrasonicator probe may be employed for a 6-minute ultrasonication program, which may involve repeated cycles of 3 minutes of pulsing (at 75% amplitude) and 30 seconds of rest. In specific embodiments for 24-probe boom egg/spore lysis, a ⅛ inch diameter ultrasonicator probe may be employed for a 6-minute ultrasonication program, which may involve repeated cycles of 3 minutes of pulsing (at 50-75% amplitude) and 30 seconds of rest. In specific embodiments for 24-probe boom cyst lysis, a ⅛ inch diameter ultrasonicator probe may be employed for a 20-minute pulsing ultrasonication program, which may involve repeated cycles of 15 seconds of pulsing (at 75% amplitude) and 15 seconds of rest. By implementing whole-plate and/or multi-probe sonication/ultrasonication programs, the lysis stage can be considered high-throughput, especially relative to preexisting techniques.

After ultrasonication, 250 μL of 20% cTAB solution pre-warmed to about 50-65° C. can be added to each well of the multi-well plate. The plate can then be incubated at 65° C. for about 30 minutes. After incubation, the plate can be centrifuged, for example at 2500×g for 7 minutes at room temperature (˜20° C.).

DNA Precipitation Stage.

In some embodiments, for example those involving enhanced microbial lysis, the supernatant formed via centrifuging the lysed cellular components, which may be about 4 mL in volume for each soil sample, can be transferred to a clean, multi-well plate without transferring any soil. After the transfer, the microbial DNA released from the lysed cells can be precipitated with isopropanol. For example, about 4 mL of cold isopropanol (maintained at about −20° C.) can be added to each aliquot of transferred supernatant containing the released microbial DNA. A lid may then be placed on top of the plate, which is then incubated at −20° C. for about 2 hours to overnight. The incubated samples can then be removed from the freezer and centrifuged at 2500×g for 7-10 minutes at room temperature (˜20° C.) to pellet the precipitated DNA. The supernatant can be removed by quickly decanting.

DNA Binding Stage:

In some embodiments, for example those not involving enhanced lysis, a DNA binding stage can be implemented in lieu of or in addition to the DNA precipitation stage. The DNA binding stage causes DNA molecules released from the lysed cells to bind with DNA-binding particles, such as the silica particles included in the DNA binding solution. The DNA binding stage may first involve preparing 1.2 mL (or about 1.2 mL) of a fresh mixture of DNA binding solution and DNA binding buffer for each soil sample, which can be added to each 8-mL well in a clean multi-well plate. Each aliquot of the fresh mixture can comprise 200 μL (or about 200 μL) DNA binding solution and 1 mL (or about 1 mL) of DNA binding buffer. The supernatant generated during centrifugation of the lysed cells can then be added to each well containing the 1.2-mL aliquot of DNA binding mixture. For each sample, the volume of supernatant removed after centrifugation and combined with DNA binding mixture may be about 4 mL. Care should be used to ensure that no soil is transferred with the supernatant. Each plate may then be covered with a paraffin film, e.g., PARAFILM®, and stacked using metal stacking plates, if necessary. Stacked plates may be connected, e.g., via clamps. The covered plates can then be incubated for about 15 minutes at room temperature on a shaker set at 200 rpm. After 15 minutes, the paraffin film can be removed, and the plates centrifuged at or about 4816×g (4700 rpm) for 5 minutes at room temperature, thereby forming a pellet comprised of DNA-bound silica and a liquid supernatant in each well. The supernatant can be removed, e.g., using a pipette, such that only the DNA-bound silica pellet remains in each well. Non-DNA components that remain associated with the silica can be removed during the washing stage.

Washing Stage:

The washing stage removes extraneous, i.e., non-DNA, soil debris, residual extraction chemicals, and cellular components, including proteins, organelles, lipids, membranes, etc., from the silica particles. The washing stage may be the same or similar for methods involving non-enhanced lysis, enhanced lysis, isopropanol-based DNA precipitation, and/or silica-based DNA binding. The washing stage may involve adding 1 mL of wash buffer to each well containing either a DNA-bound silica pellet or a pure DNA pellet, and resuspending the pellet therein. Resuspending the pellet components can involve gently shaking each plate against a table, for example, and/or swirling or pipetting up and down. For the DNA-bound silica, the resuspension can then be centrifuged at or about 1962×g (3000 rpm) for 1 minute at room temperature. For the pure DNA, the resuspension can be centrifuged at or about 2500×g for 2 minutes at room temperature. The wash buffer supernatant generated via centrifugation can be removed, and the washing step can be repeated using a fresh aliquot of 1 mL wash buffer. After a second round of centrifugation, the supernatant can again be removed via pipette. To remove as much supernatant as possible, the plates can be tipped at an angle, thereby pooling any residual liquid in each well for pipette removal. The pelleted DNA can be washed 1, 2, 3 or more times, e.g., 4 or 5 times. After the final washing step, the pure DNA and/or DNA-bound silica can be allowed to dry in a vacuum hood for up to about 1 hour at room temperature until no wash buffer remains.

Resuspension Stage:

Each dried sample of pure DNA can then be resuspended in about 100 to 150 μL of elution buffer or deionized water. Resuspension can involve pipetting up and down and/or simply placing the samples at 4° C. and allowing the DNA to resuspend. In some examples, the resuspension stage may be performed only pursuant to methods involving isopropanol-based DNA precipitation. Methods involving silica-based DNA binding may proceed directly to the elution stage.

Elution Stage:

Each dried sample of DNA-bound silica can undergo an elution stage. The elution stage elutes the isolated DNA in a buffer, such as the elution buffer described herein. The elution stage can involve adding 300 μL (or about 300 μL) of the DNA elution buffer to each well containing dried silica, and optionally pipetting to mix. The mixture of DNA elution buffer and silica can then be incubated for about 20 minutes at room temperature under gentle agitation, for example by placing the samples on a shaker set at or about 200 rpm. The plates should be kept facing up to keep the silica positioned at or near the bottom of each well. After incubation, the plates can be centrifuged at or about 4816×g (4700 rpm) for 5 minutes at room temperature to pellet the silica, thereby separating the silica from the DNA contained in the elution buffer. About 200 μL of the supernatant can then be removed from each well and placed in a fresh well. Care should be taken to avoid transferring any of the silica with the supernatant, which contains the isolated DNA.

One or more post-extraction steps may then be implemented to analyze the quantity, quality and/or identity of the isolated DNA. For example, each DNA sample can be quantified using a DNA quantification plate reader, e.g., a SPECTROSTAR® Nano reader sold by BMG Labtech. The DNA can be stored at −20° C. for long-term storage or 4° C. for shorter term storage.

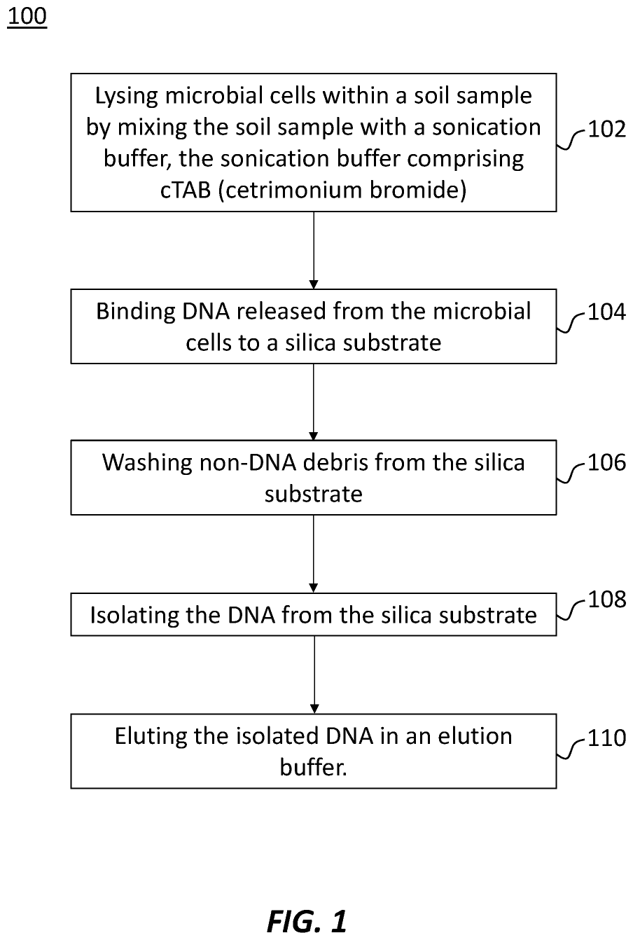

FIG. 1 is a flow diagram of an example method 100 of extracting DNA from a soil sample performed in accordance with principles of the present disclosure. The example method 100 shows the steps that may be implemented, in any sequence, to extract DNA from any soil sample by lysing the microbial cells therein, binding the released DNA, washing away non-DNA components, isolating the DNA, and optionally resuspending and eluting the DNA. In additional examples, one or more of the steps shown in the method 100 may be supplemented or omitted.

In the example shown, the method 100 begins at block 102 by “lysing microbial cells within a soil sample by mixing the soil sample with a sonication buffer, the sonication buffer comprising cTAB (cetrimonium bromide).” The method 100 continues at block 104 by “binding DNA released from the microbial cells to a silica substrate.” The method 100 continues at block 106 by “washing non-DNA debris from the silica substrate.” The method 100 continues at block 108 by “isolating the DNA from the silica substrate.” The method continues at block 110 by “eluting the isolated DNA in an elution buffer.”

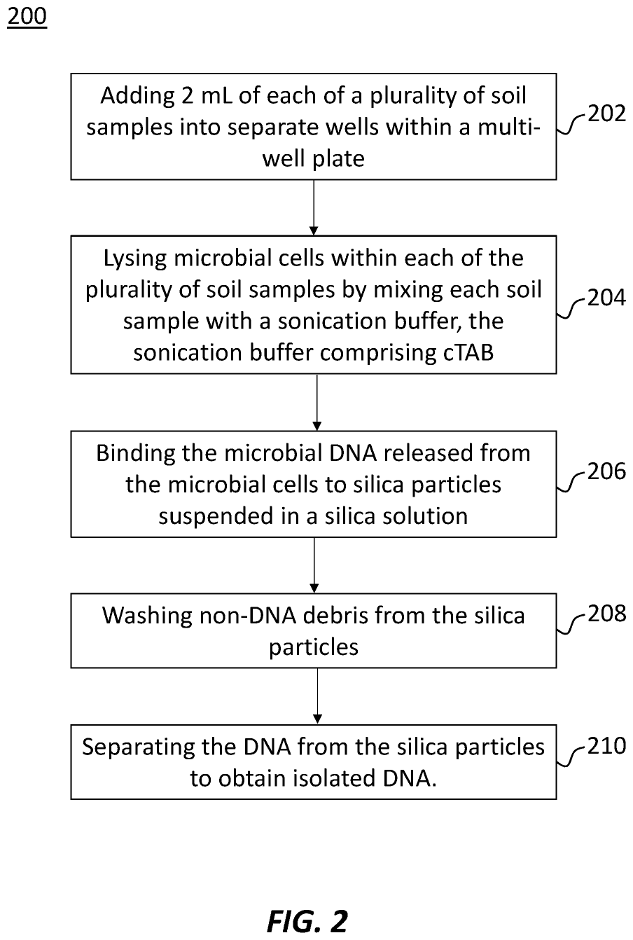

FIG. 2 is a flow diagram of an example method 200 of extracting DNA from a plurality of soil samples simultaneously. In additional examples, one or more of the steps shown in the method 200 may be supplemented, rearranged, or omitted.

In the example shown, the method 200 begins at block 202 by “adding 2 mL of each of a plurality of soil samples into separate wells within a multi-well plate.” The method 200 continues at block 204 by “lysing microbial cells within each of the plurality of soil samples by mixing each soil sample with a sonication buffer, the sonication buffer comprising cTAB.” The method continues at block 206 by “binding the microbial DNA released from the microbial cells to silica particles suspended in a silica solution.” The method continues at block 208 by “washing non-DNA debris from the silica particles.” The method continues at block 210 by “separating the DNA from the silica particles to obtain isolated DNA.”

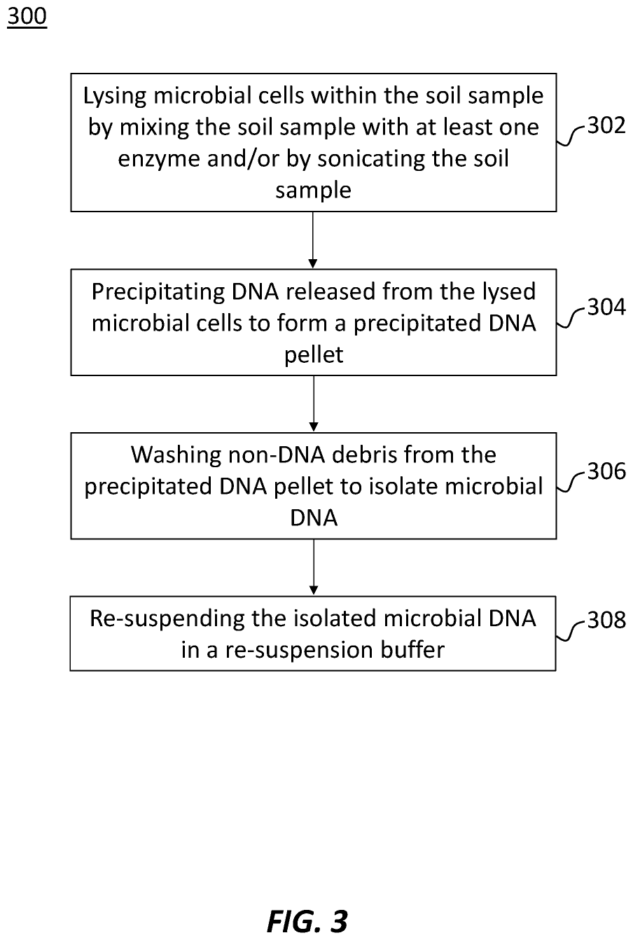

FIG. 3 is a flow diagram of another example method 300 of extracting DNA from a soil sample performed in accordance with principles of the present disclosure. In additional examples, one or more of the steps shown in the method 300 may be supplemented, rearranged, or omitted.

In the example shown, the method 300 begins at block 302 by “lysing microbial cells within the soil sample by mixing the soil sample with at least one enzyme and/or by sonicating the soil sample.” The method 300 continues at block 304 by “precipitating DNA released from the lysed microbial cells to form a precipitated DNA pellet.” The method 300 continues at block 306 by “washing non-DNA debris from the precipitated DNA pellet to isolate microbial DNA.” The method 300 continues at block 308 by “re-suspending the isolated microbial DNA in a re-suspension buffer.” The method 300 represented in FIG. 3 may be especially effective for extracting DNA present within microbial species protected by fibrous and/or proteinaceous materials. Such microbial species may include soybean cyst nematodes, or other cysts or fungal spores, which may be protected with layers or chitin and/or cellulose.

The DNA yield obtained according to the methods described herein may be approximately equal to or better than the yield obtained using preexisting commercial kits. About 1 μL of each DNA sample (in the elution buffer) may be sufficient to perform PCR. All reagents may be completely used, such that no waste remains, and all plates washed and re-used. The quantity and concentration of DNA may be suitable for a vary of settings and applications. For example, research institutions, industry laboratories, soil production laboratories, government laboratories, etc. may all implement the methods herein. DNA from various microbial species can be isolated and analyzed, including but not limited to: Bacillus anthracis, Bacillus subtilis, and Streptomyces species, along with soybean cyst nematodes and various fungal spores.

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or components used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

What is claimed is:

1. A method of extracting DNA (deoxyribonucleic acid) from a soil sample, the method comprising:
lysing microbial cells within the soil sample by mixing the soil sample with at least one enzyme and/or by sonicating the soil sample;
precipitating DNA released from the lysed microbial cells to form a precipitated DNA pellet;
washing non-DNA debris from the precipitated DNA pellet to isolate microbial DNA; and
re-suspending the isolated microbial DNA in a re-suspension buffer.

2. The method of claim 1, wherein mixing the soil sample with at least one enzyme comprises mixing the soil sample with cellulase.

3. The method of claim 1, wherein mixing the soil sample with at least one enzyme comprises mixing the soil sample with a mixture of chitinase and cellulase.

4. The method of claim 1, wherein sonicating the soil sample comprises ultrasonicating the soil sample with a sonication buffer in an ice bath.

5. The method of claim 4, wherein ultrasonicating the soil sample comprises ultrasonicating the soil sample with an ultrasonicator probe ranging in diameter from about ⅛ in. to about ¼ in.

6. The method of claim 4, wherein ultrasonicating the soil sample comprises ultrasonicating the soil sample according to a pulsating sonication program.

7. The method of claim 6, wherein the pulsating sonicating program comprises interleaving segments of pulsing and rest, the pulsing segments implemented at about 50 to about 75% amplitude.

8. The method of claim 4, further comprising adding a cTAB solution to the sonicated soil sample.

9. The method of claim 8, wherein the cTAB solution is pre-warmed to a temperature ranging from about 50° C. to about 65° C.

10. The method of claim 8, wherein the cTAB solution comprises about 20% pure cTAB.

11. The method of claim 1, further comprising wet-sieving the soil sample prior to lysing the microbial cells.

12. The method of claim 11, wherein wet-sieving the soil sample comprises mixing the soil sample with water to form a soil slurry and passing the soil slurry through one or more sieves.

13. The method of claim 12, wherein the one or more sieves comprise two stacked sieves having different pore sizes.

14. The method of claim 1, wherein the soil sample comprises between about 250 and about 750 mg of soil, inclusive.

15. The method of claim 1, wherein the soil sample comprises a 2 mL mixture of soil and water.

16. The method of claim 1, wherein washing non-DNA debris from the precipitated DNA pellet comprises re-suspending the pellet in a wash buffer and centrifuging the re-suspension.

17. The method of claim 1, wherein precipitating DNA released from the lysed microbial cells comprises admixing isopropanol with the lysed microbial cells.

18. The method of claim 17, wherein the isopropanol is maintained at a temperature of about −20° C.

19. The method of claim 18, further comprising incubating the lysed microbial cells and the isopropanol for at least about 2 hours.

20. The method of claim 19, further comprising centrifuging the lysed microbial cells and the isopropanol at about 2500×g for about 7 to about 10 minutes after incubating.
